(19) United States
(12) Patent Application Publication
(10) Pub. No.: US 2009/0068231 A1
(43) Pub. Date: Mar. 12, 2009

(54) LIVE ATTENUATED MYCOPLASMA STRAINS

(75) Inventors: Mahesh Kumar, Fort Dodge, IA (US); Muhammad Ayub Khan, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/207,698

(22) Filed: Sep. 10, 2008

Related U.S. Application Data
(60) Provisional application No. 60/993,456, filed on Sep. 11, 2007.

(51) Int. Cl. A61K 39/04; C12N 1/20; C12Q 1/32; C12Q 1/26; C12Q 1/02; C12Q 1/68

(52) U.S. Cl. 424/264.1; 435/253.1; 435/26; 435/25; 435/29; 435/6

(57) ABSTRACT

The present invention provides live, attenuated Mycoplasma bacteria that exhibit reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species. Also provided are vaccines and vaccination methods involving the use of the live, attenuated Mycoplasma bacteria, and methods for making live attenuated Mycoplasma bacteria.

### CROSS-REFERENCE TO RELATED APPLICATIONS

This application claims priority from copending U.S. provisional application No. 60/993,456, filed Sep. 11, 2007, the entire disclosure of which is hereby incorporated by reference.

### BACKGROUND OF THE INVENTION

1. Field of the Invention

The present invention relates to the fields of microbiology and immunology. More specifically, the invention relates to novel vaccines against bacterial pathogens.

2. Background Art

Mycoplasmas are small prokaryotic organisms (0.2 to 0.3 μm) belonging to the class Mollicutes, whose members lack a cell wall and have a small genome size. The mollicutes include at least 100 species of Mycoplasma. Mycoplasma species are the causative agents of several diseases in human and non-human animals as well as in plants.

In humans, for example, M. pneumoniae, is a major cause of community-acquired pneumonia (non-pneumococcal bacterial pneumonia). Another human-pathogenic Mycoplasma, M. hominis, is associated with pathological conditions in the urogenital tract of men and the upper urogenital tract of women. M. hominis has been implicated as a cause of nongonococcal urethritis, urethroprostatitis, vaginitis, endometritis, pelvic inflammatory disease, cervicitis, infertility, postpartum septicemia, pregnancy wastage, low birth weights and birth defects. Other human-pathogenic Mycoplasma species include M. genitalium (implicated in arthritis, chronic nongonococcal urethritis, chronic pelvic inflammatory disease, other urogenital infections, infertility and AIDS/HIV), M. fermentans (implicated in Arthritis, Gulf War Syndrome, Fibromyalgia, Chronic Fatigue Syndrome, Lupus, AIDS/HIV, autoimmune diseases, ALS, psoriasis and Scleroderma, Crohn's and IBS, cancer, endocrine disorders, Multiple Sclerosis and diabetes), M. salivarium (implicated in arthritis, TMJ disorders, eye and ear disorders and infections, gingivitis and periodontal diseases including cavities), M. incognitus and M. penetrans (implicated in AIDS/HIV, urogenital infections and diseases, and autoimmune disorders and diseases), M. pirum (implicated in urogenital infections and diseases, and AIDS/HIV), M. faucium, M. lipophilum, and M. buccale (implicated in diseases of the gingival crevices and respiratory tract).

M. gallisepticum and M. synoviae are responsible for significant disease conditions in poultry. M. gallisepticum, for example, is associated with acute respiratory disease in chickens and turkeys and can also cause upper respiratory disease in game birds. In addition, M. gallisepticum has been recognized as a cause of conjunctivitis in house finches in North America. With regard to M. synoviae, infection of poultry with this species leads to a decrease in body weight gain and loss of egg production.

In swine, M. hyopneumoniae is the etiologic agent of mycoplasmal pneumonia, causing significant economic loss in the swine industry due to reduced weight gain and poor feed efficiency. Infection of pigs with M. hyopneumoniae causes a chronic cough, dull hair coat, retarded growth and unthrifty appearance lasting several weeks. Characteristic lesions of purple to gray areas of consolidation, particularly in ventral apical and cardiac lobes are observed in infected animals.

M. bovis is a bovine pathogen in housed or intensively reared beef and dairy cattle. The most frequently reported clinical manifestation is pneumonia of calves, which is often accompanied by arthritis, also known as pneumonia-arthritis syndrome. Its etiological role has also been associated with mastitis, otitis, and reproductive disease or disorders of cows and bulls.

An effective strategy for preventing and managing diseases caused by Mycoplasma infection is by vaccination with live, attenuated strains of Mycoplasma bacteria. The advantages of live attenuated vaccines, in general, include the presentation of all the relevant immunogenic determinants of an infectious agent in its natural form to the host's immune system, and the need for relatively small amounts of the immunizing agent due to the ability of the agent to multiply in the vaccinated host.

Live attenuated vaccine strains are often created by serially passaging a virulent strain multiple times in media. Although live attenuated vaccine strains against certain Mycoplasma species have been obtained by serial passaging, such strains are generally poorly characterized at the molecular level. It is assumed that attenuated strains made by serial passaging have accumulated mutations which render the microorganisms less virulent but still capable of replication. With regard to attenuated Mycoplasma strains, however, the consequences of the mutations that result in attenuation (e.g., the identity of proteins whose expression pattern has been altered in the attenuated strain) are usually unknown.

Accordingly, a need exists in the art for new live, attenuated Mycoplasma bacteria that have been characterized at the proteomic level and that are safe and effective in vaccine formulations.

### BRIEF SUMMARY OF THE INVENTION

The present invention is directed to live, attenuated Mycoplasma bacteria that exhibit reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species. The live attenuated Mycoplasma bacteria of the invention can be of any Mycoplasma species. In a specific, non-limiting, exemplary embodiment, the invention provides a live, attenuated M. gallisepticum strain that exhibits reduced expression of pyruvate dehydrogenase, phosphopyruvate hydratase, 2-deoxyribose-5-phosphate aldolase, and ribosomal protein L35, relative to wild-type M. gallisepticum bacteria. According to certain embodiments of the present invention, the live, attenuated Mycoplasma bacteria of the invention are characterized by proteomic analysis as having reduced expression of one or more of the aforementioned proteins.

The present invention also provides vaccine compositions comprising the live, attenuated Mycoplasma bacteria of the invention, as well as methods of vaccinating an animal against Mycoplasma infection.

In addition, the present invention provides methods for making and/or identifying attenuated Mycoplasma clones. According to this aspect of the invention, the methods comprise subjecting an initial population of Mycoplasma bacteria to attenuating conditions, assaying individual clones for reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species, and testing the clones for virulence. Mycoplasma clones produced according to the methods of this aspect of the invention will preferably exhibit reduced expression of at least one of the aforementioned proteins and reduced virulence relative to a wild-type Mycoplasma bacterium of the same species.

### BRIEF DESCRIPTION OF THE DRAWING

FIG. 1 is a photograph of a two-dimensional (2-D) polyacrylamide gel depicting protein spots of the attenuated M. gallisepticum strain MGx+47. Circled spots numbered 19, 49, 74, 108, 114, 127, 147, 166, 175 and 225 correspond to proteins that are up-regulated in MGx+47 relative to wild-type strain R-980. Circled spots numbered 40, 68, 98, 99, 130, 136 and 217 correspond to proteins that are down-regulated in MGx+47 relative to wild-type strain R-980.

### DETAILED DESCRIPTION OF THE INVENTION

The present invention is directed to live, attenuated Mycoplasma bacteria that are suitable for use in vaccine formulations. The Mycoplasma bacteria of the present invention exhibit reduced expression of one or more of the following proteins: pyruvate dehydrogenase, phosphopyruvate hydratase, 2-deoxyribose-5-phosphate aldolase, and/or ribosomal protein L35, relative to the expression of these proteins in a wild-type Mycoplasma bacterium of the same species.

### Mycoplasma Species

The present invention is based, in part, on the surprising discovery of a new live, attenuated Mycoplasma gallisepticum vaccine strain that was demonstrated by proteomic analysis to have reduced levels of proteins such as pyruvate dehydrogenase, phosphopyruvate hydratase, 2-deoxyribose-5-phosphate aldolase, and ribosomal protein L35. (See Example 3 herein). The invention is exemplified by working examples using M. gallisepticum; however, the finding that reduced levels of pyruvate dehydrogenase, phosphopyruvate hydratase, 2-deoxyribose-5-phosphate aldolase, and ribosomal protein L35 correlates with bacterial attenuation is applicable to all species of Mycoplasma due to conservation of these proteins across Mycoplasma species.

For instance, homologues of the M. gallisepticum pyruvate dehydrogenase protein (also known as AcoA) are found in, inter alia, M. hyopneumoniae 232, M. hyopneumoniae 7448, M. hyopneumoniae J, M. florum, Mycoplasma capricolum subsp. capricolum, Mycoplasma genitalium, Mycoplasma mobile 163K, Mycoplasma mycoides subsp. mycoides SC, Mycoplasma penetrans, Mycoplasma pneumoniae, Mycoplasma pulmonis, and Mycoplasma synoviae.

Homologues of the M. gallisepticum phosphopyruvate hydratase protein (also known as Eno) are found in, inter alia, M. hyopneumoniae 232, M. hyopneumoniae 7448, M. hyopneumoniae J, M. florum, Mycoplasma capricolum subsp. capricolum, Mycoplasma genitalium, Mycoplasma mobile 163K, Mycoplasma mycoides subsp. mycoides SC, Mycoplasma penetrans, Mycoplasma pneumoniae, Mycoplasma pulmonis, Mycoplasma synoviae, Onion yellows phytoplasma, Ureaplasma urealyticum/parvum, and Aster yellows witches-broom phytoplasma.

Homologues of the M. gallisepticum 2-deoxyribose-5-phosphate aldolase protein (also known as DERA or DeoC) are found in, inter alia, M. hyopneumoniae 232, M. hyopneumoniae 7448, M. hyopneumoniae J, M. florum, Mycoplasma capricolum subsp. capricolum, Mycoplasma genitalium, Mycoplasma mobile 163K, Mycoplasma mycoides subsp. mycoides SC, Mycoplasma penetrans, Mycoplasma pneumoniae, Mycoplasma pulmonis, Mycoplasma synoviae, and Ureaplasma urealyticum/parvum.

Homologues of the M. gallisepticum ribosomal protein L35 protein (also known as Rpml) are found in, inter alia, M. hyopneumoniae 232, M. hyopneumoniae 7448, M. hyopneumoniae J, M. florum, Mycoplasma genitalium, Mycoplasma pneumoniae, and Mycoplasma pulmonis.

The above lists of homologues are intended to be illustrative and are not intended to be exhaustive, and it will be appreciated by those of ordinary skill in the art that additional homologues of M. gallisepticum AcoA, Eno, DeoC and/or Rpml exist in Mycoplasma species in addition to those listed above.

Since most Mycoplasma species express a version of pyruvate dehydrogenase, phosphopyruvate hydratase, 2-deoxyribose-5-phosphate aldolase and ribosomal protein L35, and since these proteins apparently serve homologous functions across species, it follows that reduced expression of these proteins is a defining characteristic of attenuated Mycoplasma strains as exemplified by the attenuated M. gallisepticum strain described in the Examples herein.

The attenuated Mycoplasma bacteria of the present invention may be of any Mycoplasma species. In a preferred embodiment, the attenuated bacteria are derived from animal-pathogenic Mycoplasma bacteria. As used herein, the term “animal-pathogenic Mycoplasma baceterium” means a bacterium that, in its wild-type, un-attenuated state, can infect and cause disease and/or illness in an animal. “Disease and/or illness in an animal” includes adverse physical manifestations in an animal as well as clinical signs of disease or infection indicated solely by histological, microscopic and/or molecular diagnostics.

Animal-pathogenic Mycoplasma bacteria include human- and non-human-pathogenic Mycoplasma bacteria. Human-pathogenic Mycoplasma bacteria include, but are not limited to, e.g., bacteria of the Mycoplasma species M. genitalium, M. fermentans, M. salivarium, M. hominis, M. pneumonia, M. incognitus, M. penetrans, M. pirum, M. faucium, M. lipophilum, and M. buccale. Non-human-pathogenic Mycoplasma bacteria include, e.g., avian-, porcine-, ovine-, bovine-, caprine- or canine-pathogenic Mycoplasma bacteria. Avian-pathogenic Mycoplasma bacteria include, but are not limited to, e.g., bacteria of the Mycoplasma species M. cloacale, M. gallinarum, M. gallisepticum, M. gallopavonis, M. glycophilum, M. iners, M. iowae, M. lipofaciens, M. meleagridis, and M. synoviae. Porcine-pathogenic Mycoplasma bacteria include, but are not limited to, e.g., bacteria of the Mycoplasma species M. flocculare, M. hyopneumoniae, M. hyorhinis, and M. hyosynoviae. Ovine-, bovine-, caprine- or canine-pathogenic Mycoplasma bacteria include, but are not limited to, e.g., bacteria of the Mycoplasma species M. capricolum subsp. capricolum, M. capricolum subsp. capripneumoniae, M. mycoides subsp. mycoides LC, M. mycoides subsp. capri, M. bovis, M. bovoculi, M. canis, M. californicum, and M. dispar.

### Reduced Expression of Mycoplasma Proteins

A person of ordinary skill in the art will be able to determine, using routine molecular biological techniques, whether an attenuated Mycoplasma bacterium exhibits reduced expression of one or more proteins that are normally expressed in wild-type Mycoplasma bacterial cells. Determining whether an attenuated bacterium exhibits reduced expression of a particular protein (e.g., pyruvate dehydrogenase, phosphopyruvate hydratase, 2-deoxyribose-5-phosphate aldolase, ribosomal protein L35, etc.), relative to a wild-type bacterium, can be accomplished by several methods known in the art. Exemplary methods include, e.g., quantitative antibody-based methods such as Western blotting, radioimmunoassays (RIAs), and enzyme-linked immunosorbant assays (ELISAs), in which an antibody is used which detects and binds to the protein of interest. In addition, since messenger RNA (mRNA) levels generally reflect the quantity of the protein encoded therefrom, quantitative nucleic acid-based methods may also be used to determine whether an attenuated Mycoplasma bacterium exhibits reduced expression of one or more proteins. For example, quantitative reverse-transcriptse/polymerase chain reaction (RT-PCR) methods may be used to measure the quantity of mRNA corresponding to a particular protein of interest. Numerous quantitative nucleic acid-based methods are well known in the art.

The following is a non-limiting, exemplary method that can be used for determining whether an attenuated Mycoplasma bacterium exhibits reduced expression of, e.g., phosphopyruvate hydratase. For purposes of this illustrative method, it will be assumed that the Mycoplasma bacterium is of the species M. gallisepticum, however, it will be appreciated by persons of ordinary skill in the art that this exemplary method can be applied equally to all species of Mycoplasma and can be used to assess the relative expression of any Mycoplasma protein.

First, a population of attenuated M. gallisepticum cells and a population of wild-type M. gallisepticum cells are grown under substantially identical conditions in substantially the same culture medium. Next, the two populations of cells are subjected to cell-disrupting conditions. The disrupted cells (or the protein-containing fractions thereof) are subjected, in parallel, to SDS polyacrylamide gel electrophoresis (SDS-PAGE) and then to Western blotting using an antibody which binds to the M. gallisepticum phosphopyruvate hydratase protein (such antibodies can be obtained using standard methods that are well known in the art). A labeled secondary antibody is then applied in order to provide a measurable signal that is proportional to the amount of the protein derived from the cells. If the amount of signal exhibited by the attenuated M. gallisepticum strain is less than the amount of signal exhibited by the wild-type M. gallisepticum strain, then it can be concluded that the attenuated strain exhibits reduced expression of phosphopyruvate hydratase relative to the wild-type strain. Variations on this exemplary method, as well as alternatives thereto, will be immediately evident to persons of ordinary skill in the art.

The present invention includes attenuated Mycoplasma bacteria that exhibit any degree of reduction in expression of a protein (e.g., pyruvate dehydrogenase, phosphopyruvate hydratase, 2-deoxyribose-5-phosphate aldolase, ribosomal protein L35, etc.) compared to the expression of that protein observed in a wild-type strain. In certain embodiments, the attenuated bacterium exhibits at least about 5% less expression of the protein relative to a wild-type bacterium. As an example, if a given quantity of a wild-type Mycoplasma strain exhibit 100 units of expression of a particular protein and the same quantity of a candidate attenuated Mycoplasma strain of the same species exhibits 95 units of expression of the protein, then it is concluded that the attenuated strain exhibits 5% less expression of the protein relative to the wild-type bacterium (additional examples for calculating “percent less expression” are set forth elsewhere herein). In certain other embodiments, the attenuated bacterium exhibits at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% less expression of the protein relative to a wild-type Mycoplasma bacterium. In yet other embodiments, the attenuated Mycoplasma strain exhibits no expression (i.e., 100% less expression) of the protein relative to a wild-type Mycoplasma bacterium.

In certain exemplary embodiments of the present invention, the attenuated bacteria exhibit at least 5% less expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species.

As used herein, the “percent less expression” of a particular protein exhibited by an attenuated Mycoplasma strain relative to a wild-type strain is calculated by the following formula: (A−B)/A×100; wherein A=the relative level of expression of the protein in a wild-type Mycoplasma strain; and B=the relative level of expression of the protein in the attenuated strain. Solely for the purpose of illustration, if a wild-type Mycoplasma strain exhibited 0.2500 units of expression of protein “Y”, and an attenuated strain of Mycoplasma exhibited 0.1850 units of expression of protein “Y” then the attenuated strain is said to exhibit [(0.2500−0.1850)/0.2500×100]=26% less expression of protein “Y” relative to the wild-type strain. Table 5 in Example 3 herein provides additional illustrative examples of percent less expression calculated for an exemplary attenuated strain of M. gallisepticum relative to a wild-type M. gallisepticum strain.

### Vaccine Compositions

The present invention also includes vaccine compositions comprising a live, attenuated Mycoplasma bacterium of the invention and a pharmaceutically acceptable carrier. As used herein, the expression “live, attenuated Mycoplasma bacterium of the invention” encompasses any live, attenuated Mycoplasma bacterium that is described and/or claimed elsewhere herein. The pharmaceutically acceptable carrier can be, e.g., water, a stabilizer, a preservative, culture medium, or a buffer. Vaccine formulations comprising the attenuated Mycoplasma bacteria of the invention can be prepared in the form of a suspension or in a lyophilized form or, alternatively, in a frozen form. If frozen, glycerol or other similar agents may be added to enhance stability when frozen.

### Methods of Vaccinating an Animal

The present invention also includes methods of vaccinating an animal against Mycoplasma infection. The methods according to this aspect of the invention comprise administering to an animal an immunologically-effective amount of a vaccine composition comprising a live, attenuated Mycoplasma bacterium of the invention. As used herein, the expression “live, attenuated Mycoplasma bacterium of the invention” encompasses any live, attenuated Mycoplasma bacterium that is described and/or claimed elsewhere herein. The expression “immunologically-effective amount” means that amount of vaccine composition required to invoke the production of protective levels of antibodies in an animal upon vaccination. The vaccine composition may be administered to the animal in any manner known in the art including oral, intranasal, mucosal, topical, transdermal, and parenteral (e.g., intravenous, intraperitoneal, intradermal, subcutaneous or intramuscular) routes. Administration can also be achieved using needle-free delivery devices. Administration can be achieved using a combination of routes, e.g., first administration using a parental route and subsequent administration using a mucosal route, etc.

In embodiments of the invention wherein the live, attenuated Mycoplasma bacterium is an avian-pathogenic Mycoplasma bacterium, e.g., an M. gallisepticum bacterium, the animal to which the attenuated bacterium is administered is preferably a bird, e.g., a chicken or a turkey. Where the animal is a bird, the vaccine formulations of the invention may be administered such that the formulations are immediately or eventually brought into contact with the bird's respiratory mucosal membranes. Thus, the vaccine formulations may be administered to birds, e.g., intranasally, orally, and/or intraocularly. The vaccine compositions for avian administration may be formulated as described above and/or in a form suitable for administration by spray, including aerosol (for intranasal administration) or in drinking water (for oral administration).

Vaccine compositions of the present invention that are administered by spray or aerosol can be formulated by incorporating the live, attenuated Mycoplasma bacteria into small liquid particles. The particles can have an initial droplet size of between about 10 μm to about 100 μm. Such particles can be generated by, e.g., conventional spray apparatus and aerosol generators, including commercially available spray generators for knapsack spray, hatchery spray and atomist spray.

### Methods for Making Attenuated Mycoplasma Clones

In another aspect of the present invention, the invention provides methods for identifying and/or making attenuated Mycoplasma clones. The methods according to this aspect of the invention comprise subjecting an initial population of Mycoplasma bacteria to attenuating conditions, thereby producing a putatively attenuated bacterial population. Next, individual clones of the putatively attenuated bacterial population are assayed for reduced expression of one or more proteins selected from the group consisting of pyruvate dehydrogenase, phosphopyruvate hydratase, 2-deoxyribose-5-phosphate aldolase, ribosomal protein L35, relative to a wild-type Mycoplasma bacterium of the same species. The clones that are identified as having reduced expression of one or more of the above-mentioned proteins are then tested for virulence. Clones that exhibit both reduced expression of one or more of the above-mentioned proteins and reduced virulence relative to a wild-type Mycoplasma bacterium of the same species are identified as attenuated Mycoplasma clones.

According to this aspect of the invention, the “initial population of Mycoplasma bacteria” can be any quantity of Mycoplasma bacteria. The bacteria, in certain embodiments are wild-type bacteria. Alternatively, the bacteria may contain one or more mutations. Preferably, however, the bacteria in the initial population are clonally identical or substantially clonally identical; that is, the bacteria preferably are all derived from a single parental Mycoplasma bacterial cell and/or have identical or substantially identical genotypic and/or phenotypic characteristics.

As used herein, the term “attenuating conditions” means any condition or combination of conditions which has/have the potential for introducing one or more genetic changes (e.g., nucleotide mutations) into the genome of a Mycoplasma bacterium. Exemplary, non-limiting, attenuating conditions include, e.g., passaging bacteria in culture, transforming bacteria with a genome-insertable genetic element such as a transposon (e.g., a transposon that randomly inserts into the Mycoplasma genome), exposing bacteria to one or more mutagens (e.g., chemical mutagens or ultraviolet light), etc. When bacterial cells are attenuated by passaging in vitro, the cells may be passaged any number of times, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more times in vitro.

The initial population of Mycoplasma cells, after being subjected to attenuating conditions, are referred to herein as a putatively attenuated bacterial population. Individual clones of the putatively attenuated bacterial population can be obtained by standard microbiological techniques including, e.g., serially diluting the cells and plating out individual cells on appropriate media. Once obtained, the individual clones of the putatively attenuated bacterial population are assayed for reduced expression of one or more specified proteins. Methods for determining whether an attenuated Mycoplasma bacterium exhibits reduced expression of one or more proteins that are normally expressed in wild-type Mycoplasma bacterial cells are described elsewhere herein. Exemplary methods include, e.g., RT-PCR-based methods, Western blot, etc.

Individual clones that are identified as having reduced expression of one or more proteins (e.g., pyruvate dehydrogenase, phosphopyruvate hydratase, 2-deoxyribose-5-phosphate aldolase, ribosomal protein L35) can be tested for virulence by administration of the clones to an animal that is susceptible to infection by the wild-type (unattenuated) version of the bacterium. As used herein, “an animal that is susceptible to infection by a wild-type Mycoplasma bacterium” is an animal that shows at least one clinical symptom after being challenged with a wild-type Mycoplasma bacterium. Such symptoms are known to persons of ordinary skill in the art. For example, in the case of a putatively attenuated M. gallisepticum strain that exhibits reduced expression of, e.g., pyruvate dehydrogenase, the strain can be administered to, e.g., turkeys or chickens (which are normally susceptible to infection by wild-type M. gallisepticum). Clinical symptoms of M. gallispeticum infection of poultry animals include, e.g., acute respiratory symptoms, pericarditis, perihepatitis, air sacculitis, trachea thickening, reduced weight gain, deciliation, abnormal goblet cells, capillary distension, increased numbers of lymphocytes, plasma cells and/or heterophils, and in some cases reduced egg production. Thus, if the putatively attenuated M. gallisepticum strain, when administered to a chicken or turkey, results in fewer and/or less severe symptoms as compared to a turkey or chicken that has been infected with a wild-type M. gallisepticum strain, then the putatively attenuated M. gallisepticum strain is deemed to have “reduced virulence.” Any degree of reduction in symptoms will identify the putatively attenuated strain as having reduced virulence. In certain embodiments, the putatively attenuated strain will be avirulent.

According to the present invention, a Mycoplasma clone that exhibits reduced expression of one or more proteins selected from the group consisting of pyruvate dehydrogenase, phosphopyruvate hydratase, 2-deoxyribose-5-phosphate aldolase, and ribosomal protein L35, and that exhibits reduced virulence relative to a wild-type Mycoplasma bacterium of the same species is an attenuated Mycoplasma clone.

The following examples are illustrative, but not limiting, of the method and compositions of the present invention. Other suitable modifications and adaptations of the variety of conditions and parameters normally encountered in molecular biology and chemistry which are obvious to those skilled in the art in view of the present disclosure are within the spirit and scope of the invention.

### EXAMPLES

### Example 1

### Generation of a Live, Attenuated M. Gallisepticum Strain

A new live, attenuated Mycoplasma gallisepticum strain was generated by passaging a wild-type M. galliespticum strain R980 multiple times in vitro. In particular, 0.1 mL seed material of wild-type M. gallisepticum strain R-980 was inoculated into 20 mL of modified Frey's medium (Frey et al., Am. J. Vet Res. 29:2163-2171 (1968) (also referred to herein as “MG culture medium”). The wild-type cells were grown until media color changed to bright yellow. The bright yellow cultures were subsequently used to re-inoculate fresh MG culture media as described above. The culture was passaged a total of 47 times in this manner. The resulting strain was tested for attenuation by vaccinating groups of birds followed by challenge using the wild-type M. gallisepticum. All the birds were necropsized two weeks post-challenge and mycoplasma related pathologies were observed. High passage strain (x+47) provided protection against the clinical signs associated with Mycoplasma gallisepticum infection. This attenuated M. gallisepticum strain designated MGx+47 (also referred to as “MG-P48”) was deposited with the American Type Culture Collection, P.O. Box 1549, Manassas, Va. 20108, on Jun. 19, 2007 and was assigned accession number PTA-8485.

### Example 2

### Safety and Efficacy Evaluation of a Live, Attenuated M. Gallisepticum Vaccine in Chickens

In this Example, the safety and efficacy of the new M. gallisepticum vaccine strain MGx+47 obtained in Example 1 was assessed in chickens.

Seventy one SPF white leghorn chickens were divided into seven groups as follows:

TABLE 1
Study Design

| Group | # Chickens | Vaccinated | Challenged |
|---|---|---|---|
| 1 | 11 | No | Yes |
| 2 | 10 | Yes | No |
| 3 | 11 | Yes | Yes |
| 4a | 10 | Yes | No |
| 4b | 11 | Yes | No |
| 4c | 9 | Yes | No |
| 5 | 9 | No | No |

The chickens in groups 2, 3, 4a, 4b and 4c were vaccinated with attenuated strain MGx+47 at 3.62×10⁷ CCU/mUbird, administered by coarse spray at 4 weeks of age. The chickens in groups 1 and 3 were challenged intratracheally (IT) at 7 weeks of age with 0.5 mL of Mycoplasma gallisepticum strain R at 7.74×10⁵ CCU/mL. Necropsy was performed on the chickens of groups 1, 2, 3 and 5 at 9 weeks of age, and necropsy was performed on the chickens of groups 4a, 4b and 4c at 7, 14 and 21 days post vaccination (DPV), respectively. The chickens were assessed for average weight gain, pericarditis, perihepatitis, airsacculitis, and tracheitis. The results are summarized in Table 2.

TABLE 2
Safety and Efficacy Summary
Vaccination = 3.62 × 10⁷ CFU/mL/bird
Challenge = 0.5 mL at 7.74 × 10⁵ CFU/mL

| | | | Average | | | | Airsacculitis | |
|---|---|---|---|---|---|---|---|---|
| | | | Weight Gain | | | | Score (average | Trachea |
| Group | Vaccinated | Challenged | (kg/day) | Pericarditis | Perihepatitis | Airsacculitis | of positives) | (Histology) |
| 1 | No | Yes | 0.016 | 0/11 | 0/11 | 9/11 | 3.56 | severe |
| | | | | | | | | tracheitis |
| 2 | Yes | No | 0.018 | 0/10 | 0/10 | 0/10 | 0 | normal |
| 3 | Yes | Yes | 0.017 | 0/11 | 0/11 | 2/11 | 2.5 | mixed |
| | | | | | | | | tracheitis |
| 4a | Yes | No | 0.016 | 0/9 | 0/9 | 0/9 | 0 | normal |
| 4b | Yes | No | 0.017 | 0/11 | 0/11 | 0/11 | 0 | normal |
| 4c | Yes | No | 0.017 | 0/10 | 0/10 | 0/10 | 0 | normal |
| 5 | No | No | 0.015 | 0/9 | 0/9 | 0/9 | 0 | normal |

TABLE 3
Safety Table: Histology Report of Formalin-Fixed
Chicken Tracheas from Individual
Vaccinated/Unchallenged Chickens (Group 4a, 4b and 4c)

| Time | | | Goblet | Capillary | LC/ | | Thickness |
|---|---|---|---|---|---|---|---|
| Point | Chicken | Cilia | Cells/M | Distension | PC | PMNs | (microns) |
| 7 | 1 | N | − | − | − | − | 30 |
| DPV | 2 | N | − | − | − | − | 30 |
| | 3 | N | − | − | − | − | 30 |
| | 4 | N | − | − | + | − | 30 |
| | 5 | N | − | − | − | − | 30 |
| | 6 | N | − | − | + | − | 30 |
| | 7 | N | − | − | + | − | 30 |
| | 8 | N | − | − | − | − | 30 |
| | 9 | N | + | − | − | − | 30 |
| 14 | 1 | N | − | − | − | − | 50 |
| DPV | 2 | N | + | − | − | − | 50 |
| | 3 | N | − | − | + | − | 50 |
| | 4 | N | − | − | − | − | 50 |
| | 5 | N | − | − | − | − | 50 |
| | 6 | N | − | − | − | − | 50 |
| | 7 | N | − | − | − | − | 50 |
| | 8 | N | − | − | − | − | 50 |
| | 9 | N | − | − | + | − | 50 |
| | 10 | N | − | − | − | − | 50 |
| | 11 | N | − | − | + | − | 50 |
| 21 | 1 | N | − | − | − | − | 50 |
| DPV | 2 | N | − | − | ++ | − | 110 |
| | 3 | N | − | − | − | − | 50 |
| | 4 | N | − | − | − | − | 50 |
| | 5 | N | − | − | − | − | 50 |
| | 6 | N | − | − | + | − | 50 |
| | 7 | N | − | − | − | − | 50 |
| | 8 | N | − | − | − | − | 50 |
| | 9 | N | − | − | − | − | 50 |
| | 10 | N | − | − | − | − | 50 |

TABLE 4
Efficacy Table: Histology Report of Formalin-Fixed Chicken
Tracheas from Individual Chickens

| | | | | Cap- | | | |
|---|---|---|---|---|---|---|---|
| | | | | illary | | | |
| | | | Goblet | Dis- | LC/ | | Thickness |
| Group | Chicken | Cilia | Cells/M | tension | PC | PMNs | (microns) |
| 1 | Not Vaccinated; Challenged | | | | | | |
| | 1 | − | + | ++ | ++++ | ++ | 410 |
| | 2 | +/− | − | − | + | − | 90 |
| | 3 | N | + | − | − | − | 50 |
| | 4 | − | − | ++++ | ++++ | − | 420 |
| | 5 | N | + | + | + | − | 60 |
| | 6 | − | + | ++++ | ++++ | +++ | 400 |
| | 7 | − | − | ++++ | ++++ | − | 440 |
| | 8 | − | − | ++++ | ++++ | ++++ | 280 |
| | 9 | − | + | − | − | − | 40 |
| | 10 | − | − | ++++ | ++++ | − | 260 |
| | 11 | − | + | ++++ | ++++ | +++ | 450 |
| 3 | Vaccinated and Challenged | | | | | | |
| | 1 | − | − | ++ | ++++ | − | 380 |
| | 2 | N | − | + | + | − | 40 |
| | 3 | N | − | + | + | − | 50 |
| | 4 | − | − | + | +++ | ++ | 220 |
| | 5 | N | − | + | + | − | 60 |
| | 6 | N | − | + | + | − | 60 |
| | 7 | N | − | − | − | − | 50 |
| | 8 | N | − | − | − | − | 50 |
| | 9 | N | − | + | + | − | 50 |
| | 10 | +/− | − | + | ++ | − | 140 |

| | |
|---|---|
| 5 | Not Vaccinated; Not Challenged |

| | | | | | | |
|---|---|---|---|---|---|---|
| 1 | N | − | − | + | − | 50 |
| 2 | N | − | − | + | − | 50 |
| 3 | N | − | − | − | − | 50 |
| 4 | N | − | − | + | − | 50 |
| 5 | N | − | − | − | − | 50 |
| 6 | N | − | − | + | − | 50 |
| 7 | N | − | − | − | − | 50 |
| 8 | N | − | − | + | − | 50 |
| 9 | N | − | − | − | − | 50 |

KEY TO SAFETY AND EFFICACY TABLES (TABLES 3 AND 4):
All “vaccinated” birds were vaccinated by coarse spray with vaccine strain MGx + 47 at 3.62 × 10⁷ CCU/mL/bird;
All “challenged” birds were challenged intratracheally (IT) with 0.5 mL of Mycoplasma gallisepticum strain R at 7.74 × 10⁵ CCU/mL
Time Point (in Table 3: Safety Table) = number of days after vaccination when the chickens were examined, expressed as # days post vaccination (DPV).
Cilia: “N” = normal cilia; “−” = deciliation;
Goblet Cells/M (“−” = normal goblet cells; “+” = mucus lying on the respiratory surface);
Capillary Distension (“−” = no distension or inflammation; “+” = moderate capillary distension or inflammation; “++” = severe capillary distension or inflammation);
LC/PC = Lymphocytes and Plasma cells (“−” = none; “+” = few; “++++” = numerous);
PMNs = Heterophils (“−” = none; “+” = few; “++++” = numerous);

The histology analysis of the group 2 chickens (vaccinated but not challenged) was substantially similar to that of the group 5 chickens (unvaccinated, unchallenged), demonstrating the safety of the newly generated MGx+47 vaccine strain. (See, e.g., Table 2 above).

With regard to efficacy, the group 3 chickens (vaccinated and challenged) showed significantly reduced airsacculitis compared to the group 1 chickens (unvaccinated and challenged). (See, e.g., Tables 2 and 4). In addition, as illustrated in Table 4, the group 3 chickens exhibited fewer histological signs of M. gallisepticum infection with regard to cillia, goblet cells, capillary distension, lymphocytes and plasma cells (LC/PC), heterophils (PMNs) and trachea thickness. (See Table 4).

Thus, this Example demonstrates that MGx+47 is a safe and effective live, attenuated M. gallisepticum vaccine strain.

### Example 3

### Proteomic Characterization of MGx+47 Vaccine Strain

In an effort to more precisely define the MGx+47 vaccine strain (see Examples 1 and 2) at the molecular level, a proteomic analysis of this strain was undertaken.

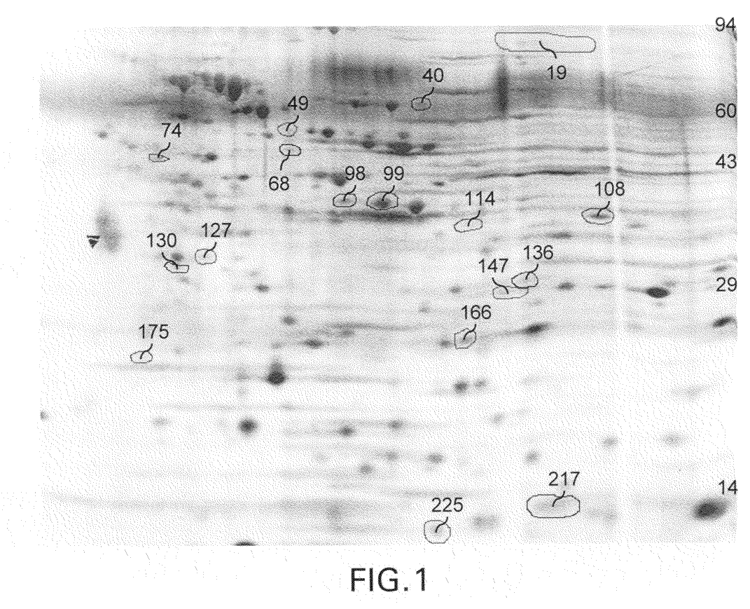

In this Example, total protein was isolated from the wild-type M. gallisepticum strain R-980 and from the newly identified vaccine strain MGx+47. Proteins from each strain were resolved by 2-dimensional polyacrylamide gel electrophoresis followed by computerized analysis of the gel images. (See FIG. 1). Protein spots were identified that were differentially expressed in the vaccine strain. Protein spots that were absent, or were expressed at significantly reduced levels, in the vaccine strain compared to the wild-type strain were excised from the gel.

Five spots were identified that were expressed at significantly lower levels in the MGx+47 vaccine strain as compared to the wild-type M. gallisepticum. Each of these protein spots were excised from the gel and enzmatically digested. Followed by peptide mass fingerprinting using matrix-assisted laser desorption/ionization-time of flight mass spectrometry (MALDI-TOF MS). The mass spectra identified for each protein spot was compared to a peptide mass database to identify the proteins and the corresponding genes that encodes them. The results of this analysis are summarized in the Table below:

TABLE 5
Summary of Proteomic Analysis of MGx + 47

| | | | Level of | | Percent |
|---|---|---|---|---|---|
| | | | expression | Level of | decrease |
| | | | in wild- | expression in | in |
| Gene | Product | Function | type MG | MGx + 47 | expression |
| acoA | Pyruvate | Required for energy | 0.1872 | 0.0858 | 54.2% |
| | dehydrogenase | production and | | | |
| | | conversion (Kreb's | | | |
| | | Cycle) | | | |
| eno | Phospho- | Catalyzes the formation | 0.0683 | 0.0173 | 74.7% |
| | pyruvate | of phosphoenol-pyruvate | | | |
| | hydratase | | | | |
| deoC | 2-deoxyribose-5- | Required for nucleotide | 0.0525 | 0.0309 | 41.1% |
| | phosphate | metabolism | | | |
| | aldolase | | | | |
| rpmI | Ribosomal | Translaction, ribosomal | 0.1171 | 0.0259 | 77.9% |
| | protein L35 | structure and biogenesis | | | |
| MGA_0621 | Hypothetical | Unknown | 0.4534 | 0.0835 | 81.6% |
| | protein | | | | |

The decrease in expression of the gene products can also be expressed in terms of “fold decrease in expression.” For example, in Table 5, strain MGx+47 can be said to exhibit 2.2, 3.9, 1.7, 4.5 and 5.4 fold decreased expression of acoA, eno, deoC, rpml, and MGA_{—}0621, respectively, relative to wild-type MG.

As indicated in Table 5, five gene products were identified that had significantly reduced expression in the live, attenuated MGx+47 vaccine strain as compared to the wild-type R-980 strain: AcoA, Eno, DeoC, Rmpl, and MGA_{—}0621 (a hypothetical protein identified under NCBI accession number NP_{—}852784). Importantly, three of these genes (acoA, eno and deoC) encode proteins involved in metabolic/energy generation pathways. In addition, homologues of AcoA, Eno, DeoC, and Rpml are found in most species of Mycoplasma, strongly suggesting that down-regulation of one or more of these gene products may be a general strategy for attenuating Mycoplasma.

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

What is claimed is:

1. A live, attenuated Mycoplasma bacterium that exhibits reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species.

2. The bacterium of claim 1, wherein said bacterium is derived from an animal-pathogenic Mycoplasma bacterium.

3. The bacterium of claim 2, wherein said animal-pathogenic Mycoplasma bacterium is a human-pathogenic Mycoplasma bacterium.

4. The bacterium of claim 3, wherein said human-pathogenic Mycoplasma bacterium is of a species selected from the group consisting of M. genitalium, M. fermentans, M. salivarium, M. hominis, M. pneumonia, M. incognitus, M. penetrans, M. pirum, M. faucium, M. lipophilum, and M. buccale.

5. The bacterium of claim 1, wherein said bacterium is derived from a non-human-pathogenic Mycoplasma bacterium.

6. The bacterium of claim 5, wherein said non-human-pathogenic bacterium is an avian-pathogenic Mycoplasma bacterium.

7. The bacterium of claim 6, wherein said avian-pathogenic Mycoplasma bacterium is of a species selected from the group consisting of M. cloacale, M. gallinarum, M. gallisepticum, M. gallopavonis, M. glycophilum, M. iners, M. iowae, M. lipofaciens, M. meleagridis, and M. synoviae.

8. The bacterium of claim 5, wherein said non-human-pathogenic bacterium is a porcine-pathogenic Mycoplasma bacterium.

9. The bacterium of claim 8, wherein said porcine-pathogenic Mycoplasma bacterium is of a species selected from the group consisting of M. flocculare, M. hyopneumoniae, M. hyorhinis, and M. hyosynoviae.

10. The bacterium of claim 5, wherein said non-human-pathogenic bacterium is an ovine, bovine, caprine or canine-pathogenic Mycoplasma bacterium.

11. The bacterium of claim 10, wherein said ovine, bovine, caprine or canine-pathogenic Mycoplasma bacterium is of a species selected from the group consisting of M. capricolum subsp. capricolum, M. capricolum subsp. capripneumoniae, M. mycoides subsp. mycoides LC, M. mycoides subsp. capri, M. bovis, M. bovoculi, M. canis, M. californicum, and M. dispar.

12. The bacterium of claim 1, wherein said bacterium exhibits at least 25% less expression of said one or more proteins relative to said wild-type bacterium.

13. The bacterium of claim 2, wherein said bacterium exhibits at least 50% less expression of said one or more proteins relative to said wild-type bacterium.

14. The bacterium of claim 3, wherein said bacterium exhibits at least 75% less expression of said one or more proteins relative to said wild-type bacterium.

15. The bacterium of claim 1, wherein said bacterium exhibits reduced expression of pyruvate dehydrogenase.

16. The bacterium of claim 1, wherein said bacterium exhibits reduced expression of phosphopyruvate hydratase.

17. The bacterium of claim 1, wherein said bacterium exhibits reduced expression of 2-deoxyribose-5-phosphate aldolase.

18. The bacterium of claim 1, wherein said bacterium exhibits reduced expression of ribosomal protein L35.

19. The bacterium of claim 1, wherein said bacterium exhibits reduced expression of pyruvate dehydrogenase, phosphopyruvate hydratase, 2-deoxyribose-5-phosphate aldolase, and ribosomal protein L35.

20. A vaccine composition comprising:
(a) a live, attenuated Mycoplasma bacterium that exhibits reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species; and
(b) a pharmaceutically acceptable carrier.

21. The vaccine composition of claim 20, wherein said bacterium exhibits at least 25% less expression of said one or more proteins relative to said wild-type bacterium.

22. The vaccine composition of claim 21, wherein said bacterium exhibits at least 50% less expression of said one or more proteins relative to said wild-type bacterium.

23. The vaccine composition of claim 22, wherein said bacterium exhibits at least 75% less expression of said one or more proteins relative to said wild-type bacterium.

24. The vaccine composition of claim 20, wherein said bacterium exhibits reduced expression of pyruvate dehydrogenase.

25. The vaccine composition of claim 20, wherein said bacterium exhibits reduced expression of phosphopyruvate hydratase.

26. The vaccine composition of claim 20, wherein said bacterium exhibits reduced expression of 2-deoxyribose-5-phosphate aldolase.

27. The vaccine composition of claim 20, wherein said bacterium exhibits reduced expression of ribosomal protein L35.

28. The vaccine composition of claim 20, wherein said bacterium exhibits reduced expression of pyruvate dehydrogenase, phosphopyruvate hydratase, 2-deoxyribose-5-phosphate aldolase, and ribosomal protein L35.

29. A method of vaccinating an animal against Mycoplasma infection, said method comprising administering to an animal an immunologically-effective amount of a vaccine composition, said vaccine composition comprising a live, attenuated Mycoplasma bacterium having reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species.

30. The method of claim 29, wherein said bacterium exhibits at least 25% less expression of said one or more proteins relative to said wild-type bacterium.

31. The method of claim 30, wherein said bacterium exhibits at least 50% less expression of said one or more proteins relative to said wild-type bacterium.

32. The method of claim 31, wherein said bacterium exhibits at least 75% less expression of said one or more proteins relative to said wild-type bacterium.

33. The method of claim 29, wherein said bacterium exhibits reduced expression of pyruvate dehydrogenase.

34. The method of claim 29, wherein said bacterium exhibits reduced expression of phosphopyruvate hydratase.

35. The method of claim 29, wherein said bacterium exhibits reduced expression of 2-deoxyribose-5-phosphate aldolase.

36. The method of claim 29, wherein said bacterium exhibits reduced expression of ribosomal protein L35.

37. The method of claim 29, wherein said bacterium exhibits reduced expression of pyruvate dehydrogenase, phosphopyruvate hydratase, 2-deoxyribose-5-phosphate aldolase, and ribosomal protein L35.

38. A method for identifying attenuated Mycoplasma clones, said method comprising:
(a) subjecting an initial population of Mycoplasma bacteria to attenuating conditions, thereby producing a putatively attenuated bacterial population; and
(b) assaying individual clones of said putatively attenuated bacterial population for reduced expression of one or more proteins selected from the group consisting of pyruvate dehydrogenase, phosphopyruvate hydratase, 2-deoxyribose-5-phosphate aldolase, and ribosomal protein L35, relative to a wild-type Mycoplasma bacterium of the same species; and
(c) testing clones identified in (b) as having reduced expression of said one or more proteins for virulence;
wherein a Mycoplasma clone that exhibits reduced expression of said one or more proteins and reduced virulence relative to a wild-type Mycoplasma bacterium of the same species is an attenuated Mycoplasma clone.

39. The method of claim 38, wherein said attenuating conditions of (a) comprise passaging said initial population of Mycoplasma bacteria at least 2 times in vitro.

40. The method of claim 39, wherein said attenuating conditions of (a) comprise passaging said initial population of Mycoplasma bacteria at least 5 times in vitro.

41. The method of claim 40, wherein said attenuating conditions of (a) comprise passaging said initial population of Mycoplasma bacteria at least 10 times in vitro.

42. The method of claim 38, wherein said attenuating conditions of (a) comprise transforming said initial population of Mycoplasma bacteria with a transposon which randomly inserts into the Mycoplasma genome.

43. The method of claim 38, wherein said attenuating conditions of (a) comprise exposing said initial population of Mycoplasma bacteria to a chemical mutagen or ultra violet light.

44. The method of claim 38, wherein said individual clones of said putatively attenuated bacterial population are assayed in (b) for reduced expression of said one or more proteins by reverse transcriptase-polymerase chain reaction (RT-PCR).

45. The method of claim 38, wherein said individual clones of said putatively attenuated bacterial population are assayed in (b) for reduced expression of said one or more proteins by Western blot.

46. The method of claim 38, wherein said clones identified in (b) are tested for virulence in (c) by administering one or more of said clones to an animal that is susceptible to infection by said wild-type Mycoplasma bacterium and comparing the clinical symptoms observed in said animals after being administered said one or more clones to the clinical symptoms of control animals that are not administered said clones.

47. The method of claim 29, wherein said vaccine composition is administered to said animal by direct injection, spray administration or drinking water administration.
